(19) United States
(12) Patent Application Publication
(10) Pub. No.: US 2007/0265658 A1
(43) Pub. Date: Nov. 15, 2007

(54) Anchoring and tethering system

(75) Inventors: Dale Nelson, Minneapolis, MN (US); Xiaoping Gu, Plymouth, MN (US); Kent Anderson, Champlin, MN (US)

Correspondence Address: NIKOLAI & MERSEREAU, P.A., 900 SECOND AVENUE SOUTH, SUITE 820, MINNEAPOLIS, MN, 55402, US

(73) Assignee: AGA Medical Corporation, Golden Valley, MN (US)

(21) Appl. No.: 11/433,302

(22) Filed: May 12, 2006

(51) Int. Cl. A61B 17/08

(52) U.S. Cl. 606/213

(57) ABSTRACT

A method and apparatus for providing safe and efficient transcatheter correction of the shape of heart chambers, valves, or other body members. The apparatus generally performs this correction by securing a tether to body surfaces. The attachment device includes an anchor comprising a wire braid and at least one clamp affixed to and constraining a portion of the wire braid, the clamp having threading for temporarily coupling the anchor to a delivery member and an internal lumen through which the tether can pass. The device also includes a locking member cooperating with the lumen to lock the tether to the clamp at a preselected position along the length of the tether.

### I. FIELD OF THE INVENTION

The present invention relates to devices and methods for improving the function of a diseased heart. Such devices and methods are particularly useful for non-invasively treating structural conditions of the diseased heart that can lead to morbidity and early death.

### II. BACKGROUND OF THE INVENTION

The human heart is a complex pumping system of contracting chambers and valves that causes blood to flow through the vascular system of the body. The healthy human heart beats on average more than 40 million times a year. Over time, disease and injury can cause damage to the heart. In a diseased heart, the chambers can become swollen and distended leading to cardiac inefficiency and heart failure. Such swelling can lead to damage to the electrical conduction paths in the heart that control its rhythm. Likewise, the annulus of the mitral valve of the heart can become distended such that the leaflets do not fully close permitting blood to regurgitate (i.e., flow in the wrong direction through the valve). Such regurgitation can result in inefficient pumping by the heart to a degree that is detrimental to patient health.

In the past, various medical, electrophysiological and surgical techniques have been used to treat such cardiac conditions. Such medical techniques have typically involved treatment using pharmaceuticals. Such pharmaceuticals do not really remedy the condition, but instead can help control the effects of the condition or prevent a worsening of the condition. For example, diuretics are available to relieve accumulation of fluids in the lungs or legs that can accompany heart failure or mitral valve regurgitation. Antibiotics are used to prevent endocarditis which can also result from such conditions. High blood pressure can exacerbate mitral valve regurgitation so drugs to treat high blood pressure have also been used.

Electrophysiologic treatments typically involve the use of a pulse generator (i.e. a pacemaker, cardioverter or defibrillator) and lead system to deliver pulse to the heart to control its rhythm.

Surgical treatments have been used to both reshape the chambers of the heart and repair or replace the valves. For example, chambers of the heart have been surgically shaped through resection of the heart tissue or by applying a patch, cuff or sleeve to the outside of the heart to constrain distended heart tissue. See, for example, U.S. Pat. No. 6,808,488 to Mortier et al. Valves have been surgically repaired through the use of annuloplasty rings, through quadrangular segmental resection of the leaflets of the valve, through shortening of the elongated cordae of the valve, or through transposition of the posterior leaflet cordae to the anterior leaflet. Human heart valves have been surgically replaced with either tissue valves from pigs or artificial mechanical valves. Heart surgery typically entails great trauma to the patient and long recovery periods. Such surgery, and particularly open heart surgery, is typically performed under general anesthesia through an incision that extends the entire length of the breastbone. The ribs are spread to expose the heart and the patient is attached to a heart-lung machine which serves as a temporary replacement for the heart during surgery. The heart must be stopped, repaired surgically, and then restarted. The risk of death during surgery is significant particularly because heart disease has often weakened the body, and particularly the heart, before such surgery is even attempted. Substantial efforts have been undertaken to find ways to treat cardiac conditions that will reduce such trauma and recovery periods.

In recent years, a variety of highly traumatic surgical procedures have been replaced with procedures that involve the use of a catheter advanced through the vascular system of the body to gain access to the heart. Balloon catheters have been used to perform angioplasty procedures as a replacement for a surgical heart bypass. Leads for cardiac rhythm management devices are now placed in the heart through the vasculature of the body rather than surgically sewn or attached to the outside of the heart. A variety of stents have been deployed via a catheter.

A variety of transcatheter deliverable devices have been developed by the assignee of the present invention to close holes in the heart. Such holes include atrial septal defects, ventricular septal defects, patent ductus arteriosus and patent foramen ovale. Recovery periods when these devices are implanted are virtually non-extent as compared to the weeks and months of recovery most patients experienced when surgical repairs were performed.

In recent years, various transcatheter approaches for non-surgical repair of heart valves and distended heart chambers have been disclosed. For example, U.S. Patent Publication No. 2001/0018611 (Solem et al) and U.S. Patent Publication No. 2005/0149182 (Alferness et al) each describe devices advanced via a catheter into the coronary sinus and deployed there to change the radius of curvature of the coronary sinus and the adjacent mitral valve annulus. In theory, changing the shape of the annulus can enable the leaflets to better close the orifice surrounded by the annulus.

While the above-referenced patent applications disclose theoretical concepts for changing the size and shape of the annulus, those skilled in the art will recognize issues that make these proposed solutions impractical for general use. First, the geometry of every heart is different. Therefore, what affect changing the radius of curvature of the coronary sinus will have on the annulus of the mitral valve cannot be accurately predicted or easily controlled. Second, even if the effect on the annulus were predictable, alignment of the biasing member in the coronary sinus would have to be precise. There is no teaching in these patent publications of how such precise alignment could be achieved. Third, the coronary sinus only surrounds about half of the mitral valve. Application of the devices shown in these patent publications may, therefore, change the shape of the annulus in a way that exacerbates mitral valve regurgitation rather than solving the problem. Fourth, the devices shown could very well lead to significant occlusion of the coronary sinus which is an essential conduit for carrying blood.

U.S. Patent Publication No. 2005/0065550 (Starksen et al) apparently attempts to overcome the problems with the coronary sinus approach for reshaping the annulus. As an alternative, that publication discloses a device that includes a plurality of hook type anchors that penetrate the annulus of the valve and cooperate with a biasing member that draws the anchors together circumferentially to tighten (i.e., reduce the size of) the valve annulus. The theory behind this concept is similar to the theory used by orthodontists to straighten teeth or correct an overbite. While in theory, the system disclosed in the Starksen et al application might work, the system is impractical given the difficulty in aligning the device, setting the hook type anchors and applying proper tension between the anchors with the biasing means to achieve the proper shape all through a catheter. There are risks of infection, damage to the muscle and thrombus formation between the various components that could lead to stroke or death.

Others have disclosed concepts for reshaping the annulus using a tether stretched across the valve and anchored at its two ends to opposing walls of the heart. Similar approaches have been disclosed for reshaping or relieving stress on the walls of a heart chamber. International Patent Publication No. WO2004/112585 (Huynh et al) shows such a device for reducing the annulus of a heart valve. One of the anchors is a stent located in the coronary sinus. The other anchor is selected from “a coil barbed anchor, a hooked anchor, and a harpoon barbed anchor”. This anchor punctures another wall of the heart. The two anchors are joined by a tension member that passes through a puncture in the wall of the coronary sinus and pulls the tissue in the location of the anchors (and, in theory, opposing sides of the valve annulus) together.

The system shown in the Huynh et al is not practical for several reasons. First, it would be extremely difficult to safely position the harpoon anchor in the coronary sinus, use it to puncture the coronary sinus, advance it across the atrium, and then securely and permanently fix it to the opposing heart wall. Even if this could be accomplished, it would be difficult to ensure that the spot where this anchor is coupled to the wall and the point where it penetrates the coronary sinus will result in tension being supplied in a manner that corrects the shape of the annulus of the valve. The risk of a tear in the coronary sinus that could lead to emergency open heart surgery cannot be overlooked. Likewise, the risk that the anchor could be pulled from the heart wall is significant.

Similar problems are inherent in the disclosure contained in U.S. Patent Publication No. 2005/0222488 (Change et al). Again, an anchor is placed in the great cardiac vein and the tether must be passed through the wall of the main vein of the heart. Also, the tether runs from a point along the great vein or coronary sinus to the fossa ovalis in the atrial septum. There is no guaranteeing that applying tension along that vector will result in proper reshaping of the annulus of the mitral valve. Chang et al does have the advantage of disclosing an anchor (modeled after applicant's septal defect occluders) that will spread the forces better and likely be more secure than the harpoon, coiled barb or hook type anchors of Huynh et al.

U.S. Pat. No. 6,764,510 to Vidlund et al discusses the need to properly align one or more tensioning members to achieve the desired effect on the valve. FIGS. 7a and 7b of this patent show a rigid elongated member 130 extending along the line of leaflet coaptation of the mitral valve. More specifically, the elongated member is positioned slightly above or slightly below the valve annulus so as to appropriately affect the valve leaflets and move them into the desired position. However, the device shown is not implanted using a catheter. Instead, the patent describes the elongated member being inserted (twice) through the wall of the left atrium and then coupled to pads 132 that rest against the outside of the heart wall. Thus, implantation requires surgical approach through the chest wall. There is nothing to suggest that either the rigid elongated member 130 or the pads 122 could be implanted using a transcatheter approach.

### SUMMARY OF THE INVENTION

A first object of the present invention is to provide methods and devices for safe and effective transcatheter correction of the shape of heart chambers and/or valves for improved performance of the heart.

A second object of the invention is to provide a tether and anchoring apparatus that can be implanted through a catheter of a relatively small diameter.

A third object of the invention is to provide such an anchor that can spread forces applied to the tissue by the anchor over a suitably large area to prevent the anchor from becoming dislodged or damaging the tissue contacted by the anchor.

A fourth object of the invention is to provide such an anchor that does not rub against or cause undue irritation to the tissue.

A fifth object of the invention is to provide a method for determining and permitting transcatheter placement of anchors at appropriate locations for achieving the desired affect on the shape of a heart chamber or valve annulus.

A sixth object of the invention is to provide a method for measuring and transcatheter adjustment of the length of a tether between two properly positioned anchors to achieve the desired affect on the shape of a heart chamber or valve annulus.

A seventh object of the invention is to provide a method for measuring and transcatheter adjustment of the forces supplied by the anchors and tether of a device to the tissue to which the device is attached.

An eighth object of the invention is to provide an anchoring and tethering device that will not detrimentally occlude a chamber or vessel.

A ninth object of the invention is to provide anchors which over a relatively short period of time will become endothelialized.

A tenth object of the invention is to provide anchoring and tethering devices that will not unduly increase the risk of embolism or stroke.

An eleventh object of the invention is to provide a method for placement of an anchoring and tethering device that does not interfere with valve operation, permits suitable blood flow past the tether, reduces the risk of nerve damage, reduces the risk of damage to electrical conducting tissues, and reduces the risk of undesirable clotting, thrombosis and infection.

A twelfth object of the invention is to provide a transcatheter technique for positioning two anchors joined together by a tether so that the tether extends along the desired vector to ensure not only improved but acceptable cardiac performance.

A thirteenth object of the invention is to provide an attachment between an anchor and the tether that permits adjustment of the length of the tether in situ.

A fourteenth object of the invention is to provide a tether and anchors that are sufficiently flexible to be advanced and positioned using a catheter and are durable enough for permanent implantation.

Another object of the invention is to provide such an anchoring and tethering device that can be safely used to treat other medical conditions such as obesity, incontinence, hernias, and wounds.

These and other objectives are met and important advantages are achieved by providing a device that includes one or more anchors preferably made of either a shape memory or super-elastic metal alloy (such as nitinol) having a first configuration for deployment through a catheter to an anchoring site comprising a hole through a wall and a second configuration for coupling the anchor to the wall at the anchoring site. More specifically, the anchor preferably comprises a braid made of at least one wire which, when in the second configuration, has a first expanded diameter portion on one side of the wall, a second expanded diameter portion on the other side of the wall, and a narrow waist that extends through the hole in the wall. The anchor further includes one or more clamps used to secure the braid. At least one of the clamps preferably has a lumen through which the tether can pass. The anchor also includes a member used to fix the tether to the anchor. This member has a first orientation which permits the tether to move longitudinally through the lumen of the clamp and a second position wherein the member grips the tether preventing such movement of the tether.

Ideally, one of the clamps will have a threaded portion which permits it to be secured to a reciprocally threaded portion at the distal end of a delivery member.

The delivery member is used to advance the anchor through the catheter to the anchoring site. The delivery member preferably has a lumen extending its entire length from its threaded portion at its distal end to its proximal end such that the tether can pass through the clamp and delivery member and out the proximal end of the delivery member. Alternating, the lumen in the delivery member extends through the threaded portion of the distal end of the delivery member and then a short distance toward the proximal end to an opening in the side wall of the delivery member. This permits the tether to pass through the clamp and lumen of the delivery member and exit the opening in the side wall of the delivery member.

When this arrangement is used, the tether exits the hole and then runs parallel to the delivery member to the proximal end of the catheter, i.e., the delivery member and a substantial portion of the tether are side by side in the catheter. This alternative embodiment makes it easier to thread the flexible tether through the lumen.

The methods of the present invention involve the following steps in different combinations:

a. determining the desired anchoring sites for the distal and proximal anchors;

b. advancing a catheter to the anchoring site for the proximal anchor;

c. advancing a puncturing tool through the catheter and puncturing a hole through the wall at the proximal anchoring site;

d. further advancing the catheter through the hole at the proximal anchoring site to the distal anchoring site;

e. advancing the puncturing tool through the catheter to the distal anchoring site, puncturing a hole through the wall at the distal anchoring site, and then withdrawing the puncturing tool from the catheter;

f. attaching tether and the delivery member to the distal anchor;

g. passing the tether through the lumen of the delivery member;

h. advancing the distal anchor through the catheter and deploying it in the hole formed at the distal anchor site;

i. releasing the delivery member from the distal anchor and withdrawing it from the catheter;

j. retracting the distal end of the catheter back to the proximal anchor site;

k. attaching the delivery member to the proximal anchor and feeding the proximal end of the tether through the proximal anchor and lumen of the delivery member;

l. advancing the proximal anchor through the catheter and deploying it in the hole formed at the proximal anchor site;

m. releasing the proximal anchor from the delivery member and withdrawing the delivery member;

n. applying tension to the tether to draw the proximal and distal anchors toward each other while monitoring the performance of the heart;

o. coupling the tether to the proximal anchor at the proper position along the length of the tether so that proper tension is supplied to satisfactorily improve performance of the heart; and

p. trimming the excess length of the tether and withdrawing the excess length of the tether and the catheter from the body. Additional steps may also be performed without deviating from the invention.

Of course, the method described above can be modified depending on the anatomy of the patient if, for example, the great vein or some already present opening in the septal wall provides an acceptable location for anchoring. Also, additional anchors and tethers can be deployed to correct chamber and valve shape and maximize cardiac performance.

### BRIEF DESCRIPTION OF THE DRAWINGS

FIG. 1 is a cross-sectional view of a human heart showing a preferred embodiment of the present invention supplying tension between the left atrial wall and the atrial septum.

FIG. 2 is a cross-section of the heart showing the heart valves.

FIG. 3 is a side view of a first embodiment of an anchor of the present invention.

FIG. 4 is a side view of a second embodiment of an anchor if the present invention.

FIG. 5 is a side view showing a pair of anchors of a third embodiment and a tether in its relaxed state.

FIG. 6 shows a delivery tool used to deploy the anchors and tether.

FIG. 7 illustrates in cross-section a first embodiment of a securement mechanism used to fix at an appropriate point along its length, a tether to an anchor;

FIG. 8 illustrates in cross-section a second embodiment of a securement mechanism used to fix, at an appropriate point along its length, a tether to an anchor;

FIG. 9 illustrates a third embodiment of a securement mechanism;

FIGS. 10a-10c are cross-sectional views of alternative securement mechanisms of the type shown in FIG. 9 taken along line A-A in FIG. 9.

FIGS. 11a-11d illustrate a fourth securement mechanism used to fix, at an appropriate point along its length, a tether to an anchor;

FIG. 12 shows a reinforcement mechanism for reinforcing an anchor;

FIG. 13 illustrates how the reinforcement mechanism of FIG. 12 is deployed;

FIGS. 14a-14e show how the tether can be fixed to an anchor using any of a variety of securement mechanisms.

### DETAILED DESCRIPTION

The human heart 1 includes four chambers, the right atrium 2, the left atrium 3, the right ventricle 4 and the left ventricle 5. The atrial septum 6 separates the right and left atria. The ventricular septum 7 separates the ventricles. The mitral valve 8 separates the left atrium 3 from the left ventricle 5. The tricuspid valve 9 separates the right atrium 2 from the right ventricle 4.

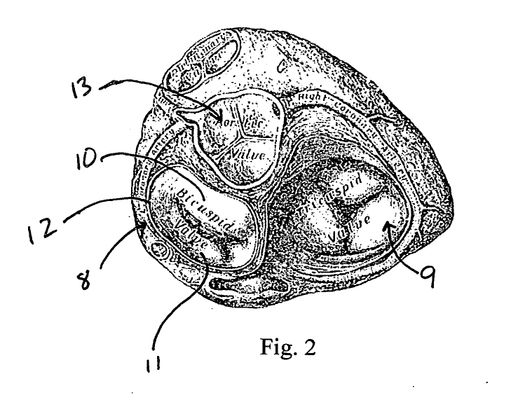

As shown in FIG. 2, the mitral valve 8, sometimes referred to as the bicuspid valve, is made up of two leaflets 10 and 11 partially surrounded by an annulus 12, a diaphanous incomplete ring around the valve. During left ventricular diastole, after pressure drops in the left ventricle 5 due to relaxation of the ventricular myocardium, the mitral valve 8 opens and blood travels from the left atrium 3 into the left ventricle 5. In a healthy heart, the mitral valve 8 closes completely and the aortic valve 13 opens so that blood is forced by contraction of the walls of the left ventricle 5 out through the aorta rather than back through the mitral valve 8 and into the left atrium 3. In a diseased heart, the atrium and ventricles can swell causing the annulus 12 of the mitral valve 8 to become distended. This, in turn, prevents the leaflets 10 and 11 from fully closing resulting in retrograde flow through the mitral valve 8 and inefficient pumping by the heart.

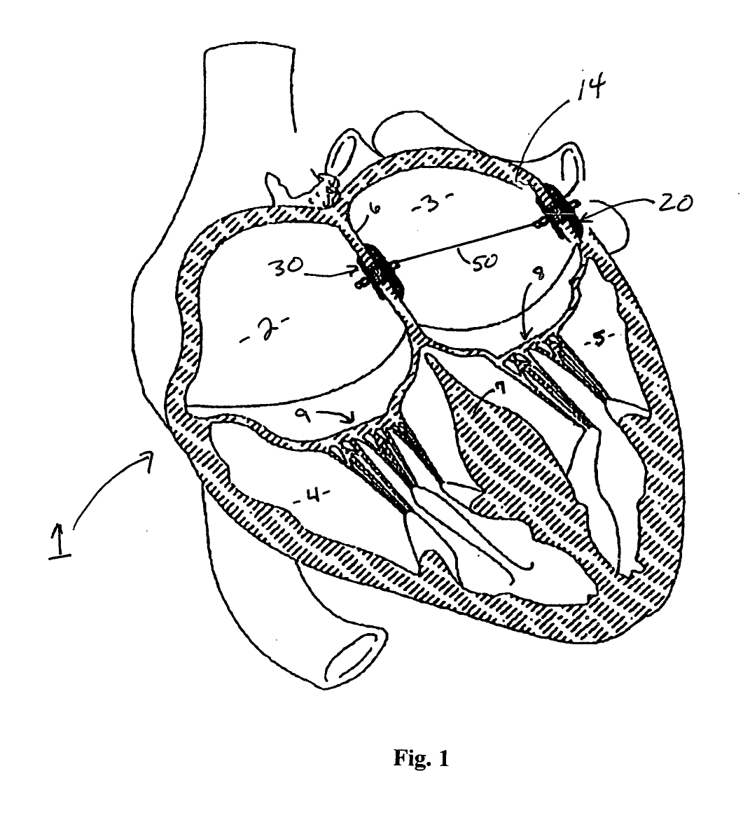

A solution for correcting swelling of the left atrium 3 and annulus 12 to provide improved closure of the mitral valve leaflets 10 and 11 is represented in FIG. 1. As shown, this solution involves locating a pair of anchors, one in the outer wall 14 of the left atrium and one in the atrial septum 6. The anchor in the atrial wall 14 is referred to herein as the distal anchor 20 and the anchor in the septum 6 is referred to as the proximal anchor 30. A tether 50 extends between and is connected to the anchors 20 and 30 to provide tension which pulls the exterior wall 14 toward the septum 6. If the requisite tension is applied by tether 50 along the correct line, the shape of the annulus 12 of the mitral valve 8 will be corrected causing the leaflets 10 and 11 to close in a satisfactory manner.

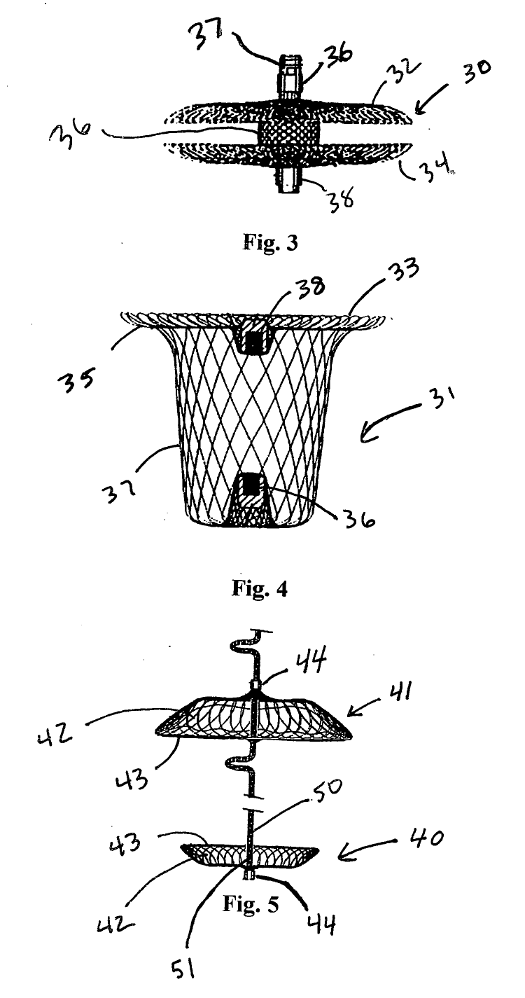

FIG. 3 shows the general construction of the anchor 30 when in its relaxed, deployed position. The anchor 20 may have either the same structure or a modified structure. As shown in FIG. 3, the anchor 30 is comprised of a wire mesh. The material used in forming the wire used in the mesh is ideally either a super-elastic or shape memory alloy. This allows the mesh to be stretched into an elongated shape for delivery via a catheter and return to the shape shown in FIG. 3 automatically when deployed from the catheter and unconstrained by the inside wall of the catheter. Portions of the anchor may also be constructed of a bioabsorbable material such as magnesium or a polymer such as PLA/GLA. Also, the bioabsorbable material will be absorbed by the body over time.

As is also shown in FIG. 3, the anchor 30 has a first expanded diameter portion 32 and a second expanded diameter portion 34 separated by a neck 36 of an appropriate length. The anchor 30 is intended to be passed through a naturally occurring or physician-created opening in the septal wall 16. When in use, the first expanded diameter portion 32 resides on one side of the wall and the second expanded diameter portion 34 resides on the opposite side of the wall. The neck 36 is located in the hole through the wall. Given this configuration, the anchor 30 not only provides a firm connection to the septal wall 6, but also occludes the opening through the septum 6. Anchor 20 similarly provides both of these functions with respect to the outer wall 14 and a hole through the outer wall.

Anchor 30 is shown as having two clamps 36 and 38. The functions performed by these clamps can, of course, be performed by a single clamp if desired. The functions of the clamps include (1) preventing the braid from unraveling; (2) providing a channel in the anchor through which the tether can pass; (3) providing one portion of a threaded connection to which a delivery member can be attached; and (4) providing sturdy engagement for the tether. Further details related to the structure of these clamps are provided below.

FIG. 4 shows an alternative anchor 31. In this embodiment, the anchor is again formed of a braided wire mesh made preferably of a super-elastic or shape memory material. The anchor has a first elongated shape permitting it to pass through a catheter and the deployed shape shown in FIG. 4. In the deployed shape, the anchor 31 has an expanded diameter portion 33 having a wall engagement surface 35 and a neck that resides in the hole in the exterior or septal wall in which anchor 33 is deployed. Anchor 31 again has a pair of clamps 36 and 38.

A further embodiment of the invention is shown in FIG. 5. The embodiment includes a first anchor 40 and a second anchor 41. Anchors 40 and 41 each comprise a braided wire mesh 42 having a tissue engaging surface 43 and a clamp 44. In FIG. 4, the tether 50 is shown as a braided cable. Use of a braided cable adds strength while maintaining the flexibility required for transcatheter delivery of the device. As discussed below, the tether may also include a central lumen 51 extending its entire length. As shown, the distal end 52 of the tether 50 is fixed to clamp 44 of the anchor 40 and passes through and is in slidable engagement with the clamp 44 of anchor 41. This slidable engagement is a temporary condition necessary for the tether 50 to be pulled tight between the two anchors 40 and 41 and so that the anchors 40 and 41 and tether 50 can cooperate to draw together the two walls to which the anchors are respectively coupled.

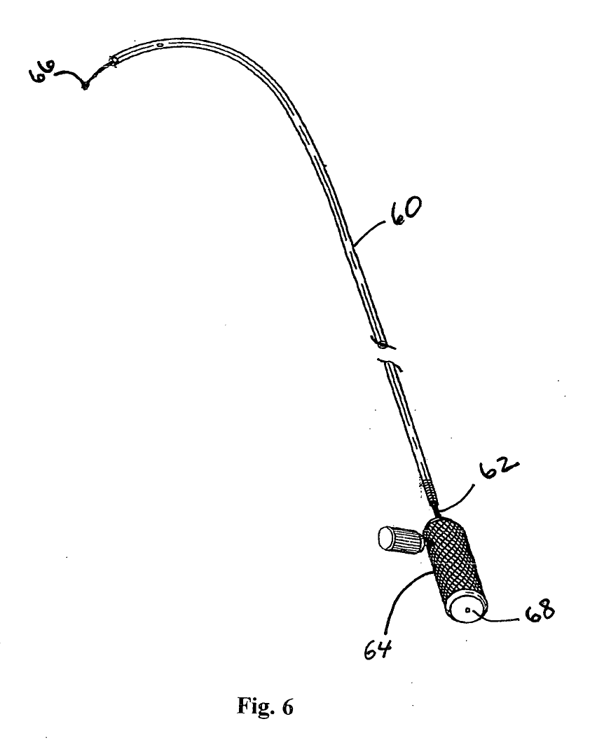

FIG. 6 shows a mechanism for delivery and implantation of the anchors. The deliver system includes a catheter 60 and a delivery mechanism 62 coupled at its proximal end to a handle 64 and at its distal end to a threaded fitting 66. The catheter 60 has a central lumen. This lumen is used for a variety of purposes. During an implantation procedure an introducer (not shown) will be used to gain access to the vasculature of the body. A guidewire (also not shown) is then passed through the introducer and vasculature to the treatment site (e.g., the right atrium of the heart). The lumen of the catheter 60 is then used to slide the catheter over the guidewire to the treatment site. The guidewire is then pulled out of the lumen.

Various tools can then be advanced through the lumen to the treatment site. Such tools could include, for example, a fiber optic cable for visualizing the treatment site or a puncturing tool for piercing the septum or other heart wall at the anchoring location. The lumen is also used for delivery of the anchors and tether, any tools needed to lock the tether to the anchors, and any tools needed to trim and withdraw excess portions of the tether.

The delivery member 62 also has a central lumen 68 that, as shown, extends the entire length of the delivery member 62. As explained below, the tether 50 (and perhaps one or more other elements) is passed through this lumen 68 during implantation of the device.

As shown in FIGS. 1 and 5, the distal end 52 of tether 50 must be fixed to the distal anchor 20/40. Various options for fixing the distal end 52 of tether 50 to the distal anchor will now be discussed with reference to FIG. 3.

First, the tether 50 can be clamped along with the ends of the wire(s) of the braid using either clamp 36 or 38. If clamp 38 is used for this purpose, the clamp 36, in addition to having threads 37, should have an internal lumen through which the tether 50 is passed. During manufacture, the distal end of tether 50 can then be fixed to clamp 38 along with the wires of the braid.

Second, both clamps 36 and 38 can have lumens extending therethrough. The distal end 52 of the tether 50 can be attached to a cap sized to seat on or in the distal end of clamp 38. The tether 50 can then be threaded through the lumens of clamps 36 and 38 and pulled until the cap seats on or in the distal end of clamp 38.

Third, it may be desirable to have the cap reside within the wire mesh. In this case, the tether is threaded through the lumen in clamp 36 and pulled until the cap seats against the distal end of clamp 36.

Fourth, the cap on the distal end of the tether 50 can be provided with threads that cooperate with matching threads on one of clamps 36 or 38. If the threads for joining the tether 59 are on clamp 36, clamp 36 will have two sets of threads, one for permanently joining the tether 50 to the clamp 36 and the other for temporarily joining the delivery member to clamp 36. The two sets of threads on clamp 36 can be provided coaxially such that one set is on the inside of the clamp and the other is on the outside of the clamp. If the threads for joining the tether 50 are on the clamp 38, the tether 50 passes through a lumen in clamp 36 so that the tether can be attached at its distal end to clamp 38.

Various other arrangements for securing the distal end of the tether 50 to a distal anchor (including those discussed for fixing the tether 50 to the proximal anchor 30/40) can be employed without deviating from the invention. Generally speaking, the tether 50 will be coupled to the distal clamp either at the time of manufacture or at some other time prior to insertion of the distal anchor into the body. Thus, any number of arrangements can successfully be used.

The range of available options is more limited for fixing the tether 50 to the proximal anchor 30. This is because the anchor 30 will not generally be fixed to the tether 50 at a specific point along its length prior to implant, but only after the proximal anchor 30 is implanted. Of course, if the physician implanting the device knew prior to implant exactly what the length of the tether 50 between anchors 20 and 30 should be, any of the securement mechanisms described above with respect to the distal anchor could also be used in securing the proximal end of the tether 50 to the proximal anchor.

Since the exact length is likely not known prior to implant, the securement arrangement used to fix the tether 50 and proximal anchor 30 together must first permit the tether 50 to be pulled through the proximal anchor 30 to take up any slack and supply the correct amount of tension, and second lock the tether 50 to the proximal anchor 30 at the desired point along the tether's length.

Preferably, the clamp(s) of the proximal anchor 30 will have lumens through which the tether 50 can slidably pass and a securement mechanism that cooperates with at least one clamp of anchor 30 to secure the tether 50 to the proximal anchor 30 at the desired point along the tether's length. If the proximal anchor 30 has two clamps 36 and 38, the most proximal clamp 36 will generally be used for this purpose.

Various securement mechanisms can be employed that meet the need to fix the proximal anchor 30 to the tether 50. For example, at least one of the clamps can be formed as a compression fitting having one or more gripping members that are biased toward a closed, gripping position. When the delivery member is attached to the fitting, the gripping members are spread permitting the tether to slide. However, as the delivery member is released, the gripping members close clamping the tether in place. Another is the use of a quick-setting cement. Still another is a cap that can slide over the tether through the catheter and cooperate with the clamp to secure the tether in place. Still another technique is to form a fitting made of a shape memory or super-elastic material that can slide over (or through) the tether and down the catheter to the location of clamp 36 which, upon warming or release from a constraint, changes shape to fix the tether 50 to the anchor 30.

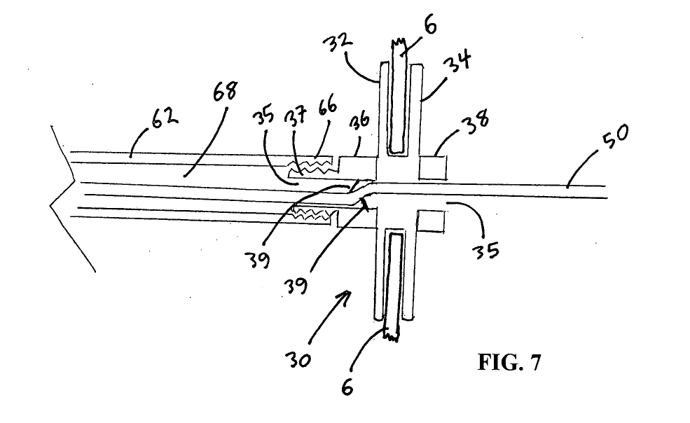

FIGS. 7-12 show in greater detail various alternative arrangements that may be used to secure the tether 50 to the proximal anchor 30. As shown in FIG. 7, the anchor 30 has a distal clamp 38 and a proximal clamp 36. Both clamps prevent the wire braid from coming apart. Both also have a central lumen 35 through which the tether 50 can pass. Clamp 36 has a threaded section 37 which cooperates with the threaded fitting 66 of the delivery member 62 to temporarily secure the anchor to the delivery member. The tether is also shown as passing through the lumen 68 of the delivery member 62.

An important feature of the embodiment shown in FIG. 7 is the set of barbs 39 located within the lumen 35 of clamp 36. The barbs 39 are angled and positioned so that the tether 50 can only be pulled in the proximal direction. The barbs 39 seat against the tether 50 preventing movement in the distal direction. More specifically, the barbs 39 must be pointed in the direction that the tether 50 is drawn to increase tension between the two anchors. While the barbs 39 could be placed in the lumen 35 of either or both clamps 36 and 38, it is typically advantageous to place barbs 39 only in clamp 36 so that tension supplied by tether 50 flattens the corresponding expanding diameter portion 32 against the septal wall 6. If the tether 50 were locked to the distal clamp 38, the tension supplied by tether 50 could pull the anchor 30 back through the hole in the wall 6.

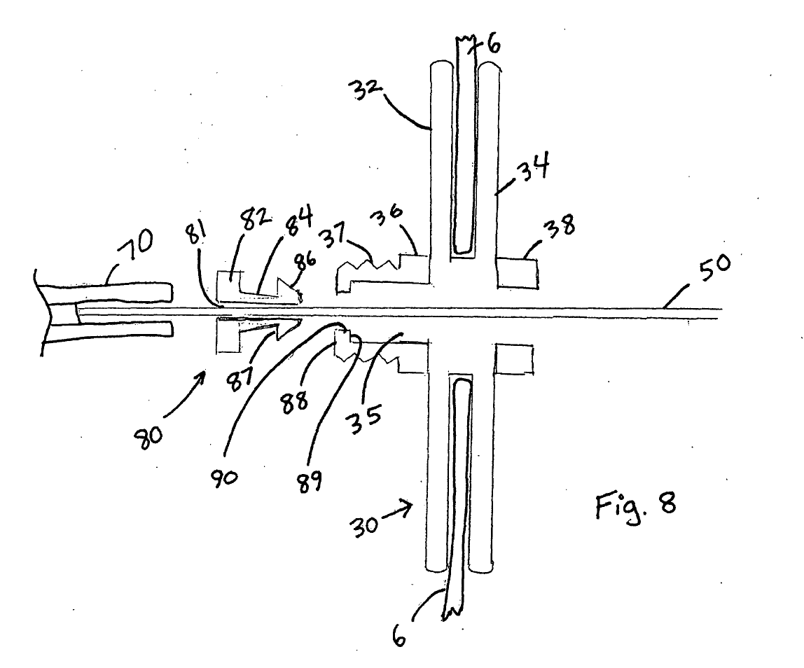

FIG. 8 shows an alternative locking arrangement. Cooperating with the clamp 36 of anchor 30 is a wedge 80 having a lumen 81 through which the tether 50 can pass. The wedge 80 has a cylindrical section 82, a locking section 84, and a tapered section 86 having a first engagement wall 87. An engagement member 88 having a second wall 89 and a third wall 90 has been added to the lumen 35 of clamp 30. When in the locking position, the first engagement wall 87 of the wedge 80 is in face-to-face registration with the second engagement wall 89 of the clamp 36. This prevents the wedge 80 from moving proximally relative to the anchor 30. Also, the locking section 84 of the wedge 80 engages the third wall 90 of the clamp 36 to close the lumen 81 of the wedge 80 against the tether 50 thus locking the tether 50 to the anchor.

The arrangement shown in FIG. 8 offers several advantages. First, the tension supplied by the tether 50 can be easily adjusted and the results of differing amounts of tension supplied can be evaluated prior to inserting the wedge 80 into the lumen 35 of clamp 36. Also, if there is a problem, the wedge 80 can be removed to permit readjustment of the tension. The wedge 80 can be made of any of a variety of polymer or metal materials.

FIG. 9 shows a modification to the wedge 80 shown in FIG. 8. In this embodiment, the wedge 80 has a cylindrical section and a tapered locking section 84. The wedge shown in FIG. 9 can be used with a clamp that does not have an engagement member 88 thus making the clamp easier to machine.

FIGS. 10a-10c show a cross-section of differing locking sections 84 of wedge 80. A lumen 81 is shown in each case. Compression cutouts 83 are also shown. The compression cutouts 83 have wall segments 85 and 87 that move toward each other as the tapered section 86 is advanced into the lumen 35 of the clamp 36. This serves to close the lumen 81 securely against the tether 50 to lock the tether 50 to the anchor.

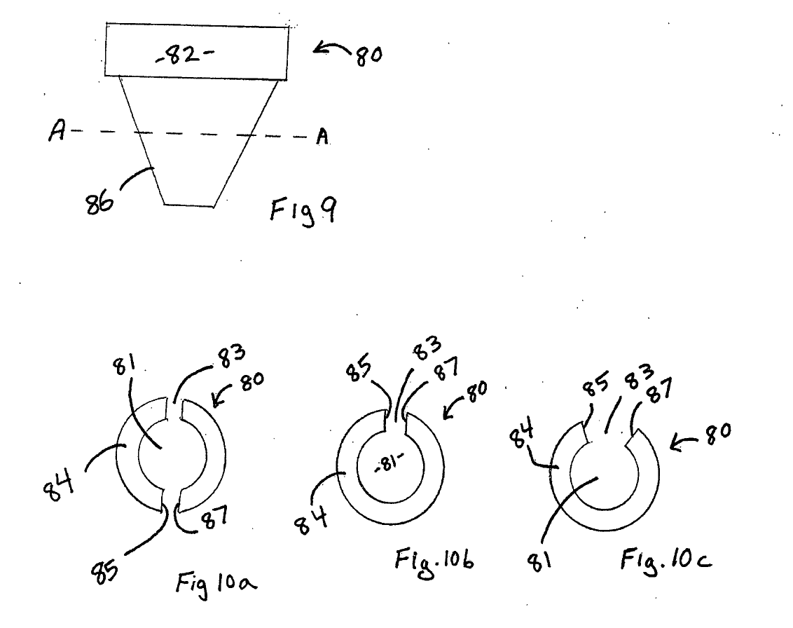

Still other alternative embodiments are shown in FIGS. 11a-11d. In these embodiments, the tether 50 has a lumen 51 such that an expansion member 100 can be advanced through lumen 51 to the location of the clamp 36 and expanded either inside the lumen 51 where it is coaxial with the lumen 35 of the clamp to fix the tether 50 to the clamp by pinching the wall of the tether 50 against the interior wall of the clamp or, as shown, just proximal to the clamp 36 so that it (and the tether at that location) has a diameter larger than the lumen 35 of the clamp 36. When the tether 50 is pulled from the distal side of the clamp 36, the section of tether 50 expanded by the expansion member 100 engages the distal surface of clamp 36 to prevent the tether 50 from being pulled through the lumen 35 toward the distal anchor. With this arrangement, the intersection between the lumen 35 and wall of the anchor 36 that engages the expanded portion of the tether 50 should be tapered so that there are no sharp edges that could cut the tether. The expansion member 100 could be in the form of a balloon that is inflated. The expansion member can also comprise various mechanical arrangements such as, for example, a self-expanding structure made of a super-elastic or shape memory material. The expansion member 100 can also be a permanent structure or simply be used to change the shape of the tether 50 and then deflated and removed as suggested by FIGS. 11c and 11d.

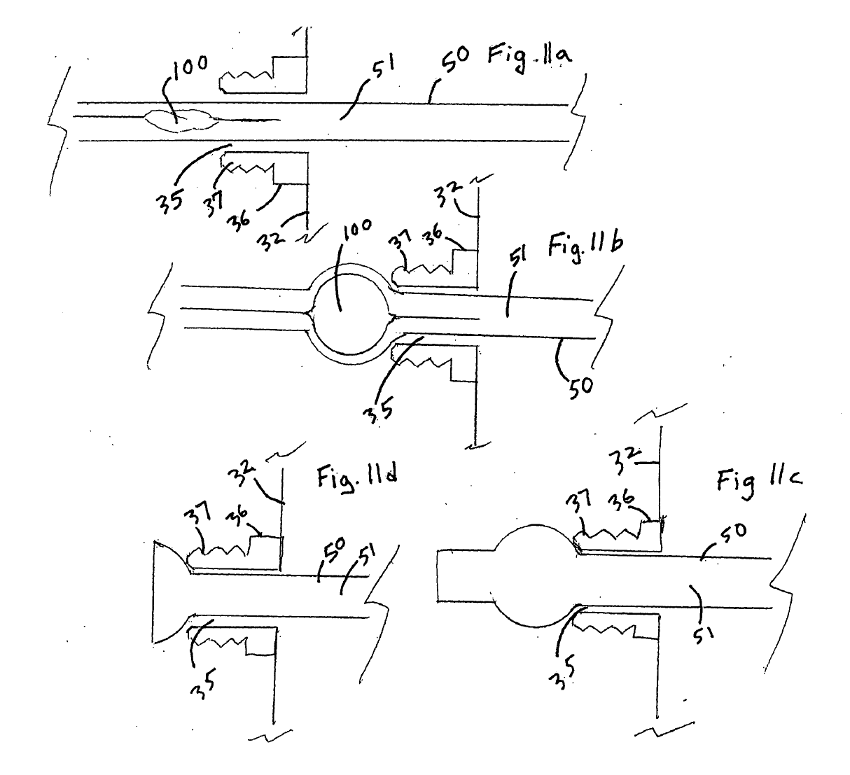

From the foregoing, it should be clear that the tension applied by the tether 50 must be less than that required to pull either of the anchors through the hole in the wall to which the anchor is attached. Therefore, in some cases it may be desirable to reinforce the expanded diameter portion on the distal side of wall 14 and the expanded diameter portion on the proximal side of wall 6 after they are in place, but before tension is applied via the tether 50. FIGS. 12-13 show how this could be done.

By way of example, FIG. 12 shows an annular circumferential ring 110 positioned within the proximal expanded diameter portion 32 of anchor 30. Once in place, ring 110 prevents the circumference of the expanded diameter portion 32 from contracting. In fact, the ring 110 preferably continually applies an urging force outwardly against the circumference of the expanded diameter portion 32. The ring 110 is preferably made of nitinol or some other super-elastic or shape memory material. As shown in FIG. 13, the ring can be straightened for passage through a lumen (such as the lumen of the delivery member) for deployment into the expanded diameter portion of the anchor. Upon deployment, the ring 110 returns to its ring shape and forces the expanded diameter portion of the anchor outwardly to prevent the expanded diameter portion from being pulled through the hole in the wall of the heart as tension is applied by the tether 50.

Use of the devices described above offers many advantages over surgical procedures used today. These advantages relate to ease of use, less trauma to the patient, and reduced recovery time. To better understand these advantages, the procedure for implementing a device made in accordance with the present invention will now be discussed.

Prior to undertaking the implant procedure, the physician will, of course, carry out various diagnostic procedures to assess whether and how use of the device might help the patient. If mitral valve regurgitation is to be treated, such diagnostic procedures would typically include radiologic study of the patient's heart to assess the degree of the problem, the geometry of the valve, and the geometry of surrounding heart structures. Such diagnostic procedures would also typically include mapping the conduction paths through the tissue of the heart. The physician will then assess the data to determine where the anchors can be placed so the tether applies appropriate tensioning to correct the problem with valve closure and does not interfere with conduction through the tissue.

The physician will also take into account the location of other structures to ensure that neither of the anchors nor the tether interferes with the operation of those structures. In the event the patient has naturally occurring, but unnecessary and often undesirable openings through the septum such as an ASD, VSD, PFO or the like, the physician considers whether the location of these openings would be suitable for anchoring.

After careful planning, the physician is ready to begin the implantation of the device. An introducer is used to gain access to the vasculature of the body. The distal end of a guidewire is inserted through the introducer and vessels until it reaches the superior vena cava at which point it is advanced through the superior vena cava into the right atrium. If the patient has an already existing opening through the septum, the guidewire can be further advanced through that opening into the left atrium. If there is no such naturally occurring opening available, the distal guidewire is left in the right atrium.

Next, a catheter 60 is advanced over the guidewire until the distal end of catheter 60 reaches the right atrium (or in the case of an already existing opening) through the septum, the left atrium. The guidewire is then removed so that the lumen of catheter 60 can be used to gain access to the heart with other devices.

One or two holes will need to be punctured to permit implantation of the anchors. Thus, the catheter is used to direct a puncturing tool to the anchor site(s). If there is not already a suitable opening in the septal wall, the desired location for this opening is identified and the puncturing tool is used to create an opening of an appropriate size. The catheter is then advanced through this first opening to the site for the other anchor and a second opening may be punctured through the tissue. The puncturing tool is then pulled back out through the catheter.

The next step is to implant the distal anchor 20 with the tether 50 secured to it. This is done via the catheter 60 using the delivery member 62. The tether 50 is pulled through the lumen 68 of the delivery member 62 and the delivery member 62 is then coupled to the distal anchor 20 using the threads of clamp 36 of the anchor and the threaded fitting 66 of the delivery member. The anchor 20 is then passed through the lumen of catheter 60 by pushing on the delivery member until it reaches the second hole. Preferably, the distal end of the catheter 60 is advanced a short distance through and past the second hole. The distal expanded diameter portion 34 is then deployed from the catheter on the distal side of the wall. The catheter is then retracted back through the hole where the rest of anchor 20 is deployed. When fully deployed, opposing surfaces of expanded diameter portions 32 and 34 contact opposite sides of the wall. Also, the neck 36 expands to engage the tissue surrounding the opening cut through the wall. The anchor 20 thus occludes the opening preventing blood from flowing out the opening cut in the wall. The anchor 20 also secures the distal end of the tether 50 in place. If desired, a reinforcing member 100 is passed through the lumen 68 of the delivery member 62 and into the distal expanded diameter portion 34 of anchor 20 where it assumes a ring shape and provides an outward force against the circumference of expanded diameter portion 34. The delivery member is then unscrewed from the anchor 20 and fully retracted from the catheter as the catheter is left in place.

Next, the proximal anchor 30 is implanted. To do so, the delivery member 62 is attached to clamp 36 of anchor 30. The proximal end of tether 50 is then inserted through the lumens in clamps of the anchor 30 and through the lumen in the delivery member. The anchor 30 is advanced through the lumen of catheter 60 to the distal end of the catheter 60 which still preferably resides in the left atrium. The distal portion 34 of anchor 30 is pushed out of the catheter and permitted to expand and deploy. The assembly is pulled back until the wall contacting surface of expanded diameter portion 34 engages the distal side of the septal wall 6. The catheter 60 is retracted further permitting the proximal expanded diameter portion 36 to deploy in the right atrium and the neck 36 to move outwardly into contact with the tissue surrounding the opening in the septal wall. In this way, the anchor 30 occludes the opening in the septal wall preventing blood flow through the opening. If desired, a reinforcing ring 110 can be deployed in expanded diameter portion 36 of anchor 30 via the lumen 68 of the delivery member 62.

At this point, the physician is ready to apply tension to the tether 50 to relieve any slack in the tether between the two anchors and also to pull tissue to which the distal anchor 20 is attached toward the proximal anchor 30. Radiological visualization and other assessment tools can be used at this point to determine what amount of tension will provide the greatest benefit in terms of proper valve closure and maximum cardiac efficiency. The tether can be temporarily clamped in place outside the body while such assessments are made.

Once the assessments are completed and the tether is in the desired position with respect to proximal anchor 30, the permanent securement mechanism is deployed, and the delivery member is detached from the anchor 30 and retracted. Of course, the permanent securement mechanism may also be deployed after the delivery member has been detached. A tether cutting tool is advanced through the catheter into the right atrium to cut the tether proximally of the clamp 36 and permanent securement mechanism. The cutting tool, excess tether material and catheter are then removed essentially completing the procedure.

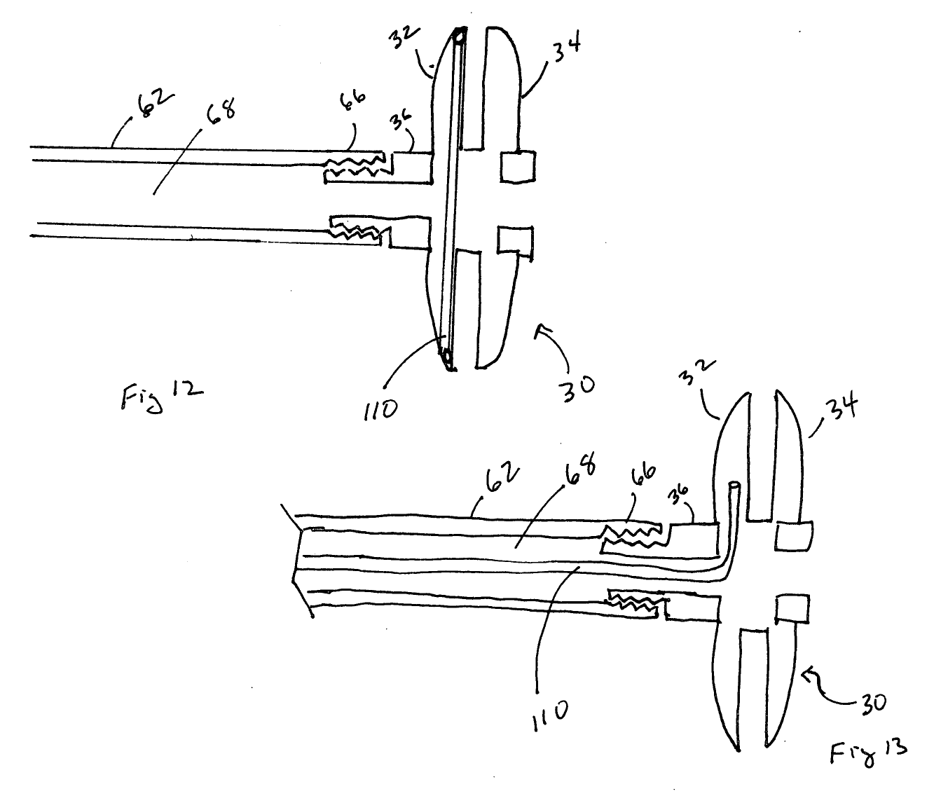

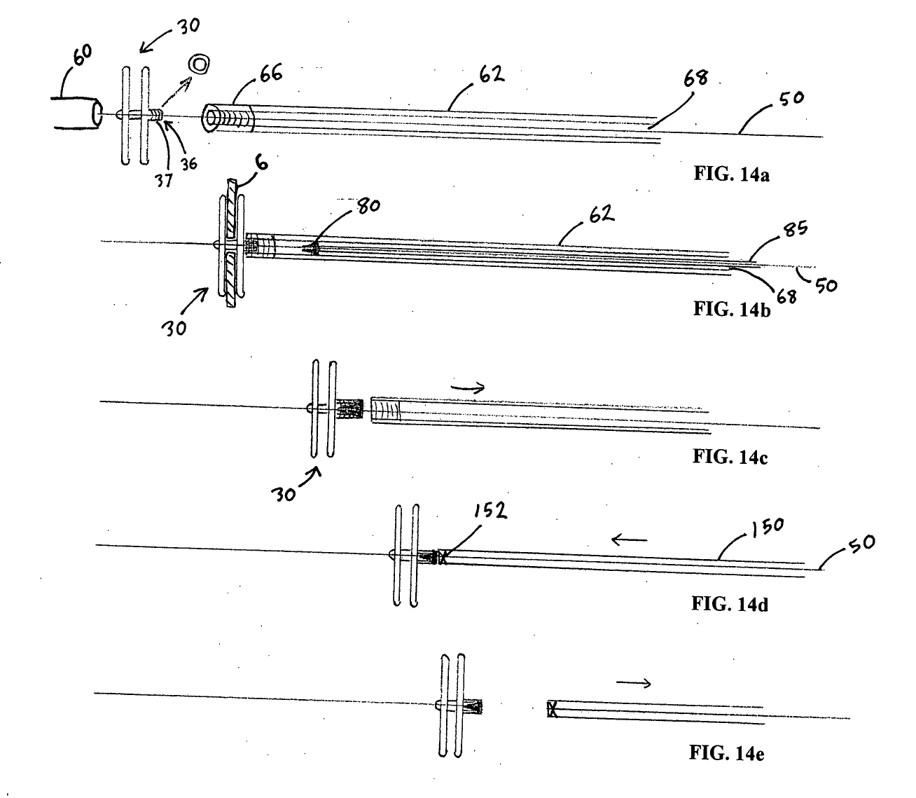

FIGS. 14a-14f are provided to help explain how the tether 50 is fixed to the anchor 30 at the correct point along the length of tether 50 to supply the correct amount of tension between the two anchors.

In FIG. 14a, the distal anchor (not shown) is already implanted with the tether 50 extending proximally through and out the proximal end of the delivery catheter 60. As shown, the proximal end of the tether 50 has been threaded through the anchor 30 and through the lumen 68 of the delivery member 62. The threaded connection between the threads 37 of proximal clamp 36 of the anchor 30 and fitting 66 of the delivery member 62 is used to temporarily join the anchor 30 to the end of the delivery member 62. The delivery member is then used to push the anchor 30 through the catheter 60 to the hole in the septum 6 where the anchor is deployed.

FIG. 14b shows the anchor 30 positioned in the hole through the septum 6. Also shown is a wedge 80 and a pusher 85 having a lumen through which the tether 50 has been threaded. The distal end of the pusher 85 engages the wedge 80 to advance the wedge 80 through the lumen 68 of the delivery member 62 toward the anchor 30. At this point, the physician can still adjust the tension supplied by the tether 50 because the tether 50 is not yet locked to the anchor 30. When the physician has determined the desired point along the tether's length, the physician temporarily clamps the tether 50 to the delivery member 62 outside of the body of the patient to restrict movement between the tether 50 and the delivery member 62. The physician then can pull slightly on the delivery member 62 while pushing on the pusher 85 to further advance the wedge 80 and to force the wedge 80 into the lumen of clamp 36 and thus lock the tether 50 to the clamp 36 at the desired point along its length.

Next, the delivery member 62 is decoupled from the anchor 30 and removed from the patient as shown in FIG. 14c. After removal of the delivery member 62, the proximal end of the tether 50 is passed through an elongated tool 150 having a cutting head 152 at its distal end. See FIG. 14d. The elongated cutting tool is then advanced through the delivery catheter 60 (not shown) until it reaches the clamp 36 of anchor 30. The cutting tool is then actuated to cut the tether 50. The unused portion of the tether 50, the cutting tool 150 and the delivery catheter 60 are then removed from the patient as indicated in FIG. 14e.

Within the first few weeks following the procedure, the anchors will endothelialize further enhancing occlusion and anchoring and further reducing the risk of thrombosis. The risk of thrombosis can be further reduced by forming the components out of or coating the components with a non-thrombogenic material.

The foregoing discussion is not intended to be limiting. Instead, the following claims define the scope of this invention.

What is claimed is:

1. An apparatus for securing a tether to a body surface comprising:
an anchor comprising a wire braid and at least one clamp affixed to and constraining a portion of the wire braid, the clamp having threading for temporarily coupling the anchor to a delivery member and an internal lumen through which said tether can pass; and
a locking member cooperating with said lumen to lock the tether to the clamp at a preselected position along the length of the tether.

2. The apparatus for securing a tether to a body surface of claim 1 wherein a portion of the anchor is made from a shape memory or super-elastic alloy.

3. The apparatus for securing a tether to a body surface of claim 2 wherein the body surface is part of a human heart.

4. The apparatus for securing a tether to a body surface of claim 3 wherein the anchor includes a first expanded diameter portion on one side of the body surface, a second expanded diameter portion found on the opposing side of the body surface, and a narrow waist portion that passes through the body surface and joins the expanded diameter portions.

5. The apparatus for securing a tether to a body surface of claim 4 wherein the tether is a flexible braided cable.

6. The apparatus for securing a tether to a body surface of claim 1 wherein the anchor is made from a bioabsorbable material.

7. The apparatus for securing a tether to a body surface of claim 1 wherein the locking member comprises at least one barb member projecting from a wall of said lumen of said clamp to lock the tether to the clamp.

8. The apparatus for securing a tether to a body surface of claim 1 wherein the locking member comprises a wedge member that cooperates with said lumen of said clamp to lock the tether to said clamp.

9. The apparatus for securing a tether to a body surface of claim 1 wherein the tether contains a hollow lumen.

10. The apparatus for securing a tether to a body surface of claim 9 wherein the locking member comprises an expansion member coaxially located within the hollow lumen of the tether capable of expanding the outer diameter of the tether to lock the tether to the clamp so that the tether cannot move relative to the clamp at least in one direction.

11. A method of securing a tether to a body surface having a hole comprising the steps of:
a. providing an anchor comprising a wire braid and at least one clamp affixed to and constraining a portion of the wire braid, said clamp having a lumen and a threaded section;
b. providing a delivery member having a lumen and threads that cooperate with the threaded section of said clamp to temporarily attach said anchor to said delivery member;
c. threading the tether through said lumen of said clamp and said lumen of said delivery member;
d. using the delivery member to advance the anchor to a hole in the body surface and attach the anchor to the body surface at the location of said hole in said body surface;
e. inserting a locking member into said lumen of said clamp to lock said tether to said clamp.

12. A method of securing a tether between first and second body surfaces of a patient comprising the steps of:
a. determining the desired anchoring site in a first body surface for a proximal anchor and in a second body surface for a distal anchor, said proximal anchor comprising a wire braid and at least one clamp coupled to and constraining a portion of said wire braid, said clamp having a lumen;
b. advancing the distal end of a catheter from outside the body to a proximal anchoring site in the first body surface for the proximal anchor and, if there is no hole through the first body surface at the proximal anchoring site, advancing a puncturing tool through the catheter and puncturing a hole through the first body surface at the proximal anchoring site;
c. advancing the catheter through the hole at the proximal anchoring site to a distal anchoring site in a second body surface for said distal anchor and if there is no hole through the second body surface at the distal anchor site for the distal anchor, advancing the puncturing tool through the catheter to the distal anchoring site, puncturing a hole through the wall at the distal anchoring site, and then withdrawing the puncturing tool from the catheter;
d. providing a tether having a distal end and a proximal end and attaching the distal end of said tether to the distal anchor;
e. passing the distal anchor through the catheter to the distal anchor site and attaching the distal anchor to the distal anchor site while retaining the proximal end of the tether outside the body;
f. retracting the distal end of the catheter back to the proximal anchor site;
g. threading the proximal end of the tether through the lumen of the clamp of the proximal anchor;
h. advancing said proximal anchor along the tether through the catheter and deploying it in the hole at the proximal anchor site;
i. applying tension to the tether to draw the proximal and distal anchors and the body surfaces to which they are attached closer to each other while monitoring the effect of such tension on the patient;
j. coupling the tether to the proximal anchor at the proper position along the length of the tether so that proper tension is supplied using a securement member that cooperates with said lumen of said clamp or said proximal anchor; and
k. trimming the excess length of the tether and withdrawing the excess length of the tether and the catheter from the body.
